(19) United States
(12) Patent Application Publication
(10) Pub. No.: US 2024/0049961 A1
(43) Pub. Date: Feb. 15, 2024

(54) Method for Vision Acuity Assessment based on Color Vision

(71) Applicant: VirtuOptica Ltd., Karmiel (IL)

(72) Inventors: Abraham Sela, Karmiel (IL); Andrey Markus, Karmiel (IL); Ori Raviv, Karmiel (IL)

(21) Appl. No.: 18/260,338

(22) PCT Filed: Dec. 10, 2021
(86) PCT No.: PCT/IL2021/051479

§ 371 Date: Jul. 5, 2023

Related U.S. Application Data
(60) Provisional application No. 63/133,885, filed on Jan. 5, 2021.

(51) Int. Cl. A61B 3/06; A61B 3/02; A61B 3/00

(52) U.S. Cl. CPC A61B 3/066; A61B 3/022; A61B 3/0041

(57) ABSTRACT

A method for carrying out an eye examination on an examinee is provided. The method includes displaying on a display at least one visual stimulus including a first visual element having a first color, a second visual element having a second color, and background having a third color, wherein the first, second and third colors are selected such that contrast between a component of the first color and the third color is the same as contrast between a component of the second color and the third color. The method further includes receiving indication from the examinee regarding detection of at least one of the first and second colors and assessing vision of the at least one eye in accordance with the indication and the contrast.

### FIELD OF INVENTION

The presently disclosed subject matter relates to an eye examination method and apparatus therefor, in general, and in particular to a method and apparatus for performing vision acuity assessment such as eye examination.

### BACKGROUND

A traditional eye examination is a series of tests performed by an ophthalmologist (medical doctor), optometrist, or orthoptist assessing vision and ability to focus on and discern objects, as well as other tests and examinations pertaining to the eyes. Health care professionals often recommend that all people should have periodic and thorough eye examinations as part of routine primary care, especially since many eye diseases are asymptomatic.

Currently, available eye examination techniques typically consist of a two-stage process, wherein a trained specialist performs an objective eye examination using an Autorefractor and a completely subjective examination using a Phoropter, wherein the examinee is required to perform a quality judgment between pairs of visual stimuli under different visual conditions, set manually by means of a Phoropter.

### SUMMARY OF INVENTION

There is provided in accordance with an aspect of the presently disclosed subject matter a method for carrying out an eye examination on an examinee. The method includes providing a display configured for displaying a visual stimulus and forming a visual path between the display and at least one eye of the examinee. The method further includes displaying on the display at least one visual stimulus including a first visual element having a first color, a second visual element having a second color, and background having a third color, wherein the first, second and third colors are selected such that contrast between a component of the first color and the third color is the same as a contrast between a component of the second color and the third color. The method further includes receiving an indication from the examinee regarding detection of at least one of the first and second colors, and assessing vision of the at least one eye in accordance with the indication and the contrast.

The step of displaying at least one visual stimulus can include displaying a series of visual stimuli wherein the contrast has a predetermined value for each one of the visual stimulus, and wherein the step of assessing vision includes assessing the contrast of the visual stimulus at which at least one of the first and second colors is detected.

Each one of the visual stimulus can include an orientation having an axis, and wherein the step of displaying visual stimulus includes displaying the visual stimulus at various orientations and the step of assessing vision further includes assessing the axis of the orientation of a visual stimulus of which at least one of the first and second colors are detected.

The first and second visual elements can include lines, wherein for each one of the visual stimulus the orientation includes disposing the lines at a predetermined angle with respect to the background.

The series of visual stimuli can include a plurality of visual stimulus such that the lines are disposed at a plurality of angles between 0°-180°.

The first, second and third colors can be selected such that difference between intensity of the component of the first color and intensity of the third color is the same as difference between intensity of the component of the second color and intensity of the third color.

The first, second and third colors can be determined with RGB parameters, and are selected such that for the first color a first parameter of the RGB parameters includes a first value, and second parameter of the RGB parameters includes a second value, and for the second color a first parameter of the RGB parameters includes the second value and second parameter of the RGB parameters includes the first value, and wherein for the third color a first and second parameter of the RGB parameters includes a third value which is an average between the first and second values.

The first and second elements can be parallel lines disposed such that the lines alternating between the first color and the second color.

The first element can include a first line in the first color having a first width and the second element includes a second line in the second color having a second width, wherein the second line is disposed on top of the first line and wherein the second width is smaller than the first width.

The visual path can include a lens having a spherical power, and wherein the step of assessing vision is in accordance with the spherical power.

The method can further include modifying the spherical power in accordance with the indication and wherein the step of displaying the visual stimulus includes displaying a plurality of visual stimulus for one or more spherical powers.

The visual stimulus can include displaying a plurality of visual stimulus each of which having characteristics corresponding to vision through a lens having a certain spherical power.

The characteristics can include a blurring level corresponding to vision via a predetermined spherical power.

The characteristics can include optical resolution level corresponding to vision via a predetermined spherical power.

The method can further include collecting dataset including axis of each visual stimulus, value of a spherical power, and the indication, the dataset is manipulated by a selected mathematical formula to obtain a required optical correction.

There is provided in accordance with another aspect of the presently disclosed subject matter a method for carrying out an eye examination on an examinee. The method includes providing a display configured for displaying a visual stimulus and forming a visual path between the display and at least one eye of the examinee. The method further includes displaying on the display at least one visual stimulus including a first visual element, a second visual element, and background, wherein contrast between the first visual element and the background is the same as contrast between the background and second visual element. The method further includes rotating the first and second elements with respect to the background modifying thereby orientation of the first and second visual elements with respect to the background, receiving an indication from the examinee regarding detection of at least one of the first and second elements, and assessing vision of the at least one eye in accordance with the indication and the contrast.

The orientation can have an axis, and wherein the step of displaying visual stimulus includes displaying the first and second visual elements at various orientations and the step of assessing vision further includes assessing the axis of the orientation when the first or second visual elements are detected.

The method can further include modifying level of the contrast and wherein the step of assessing vision further includes assessing level of the contrast when the first or second visual elements are detected.

### BRIEF DESCRIPTION OF THE DRAWINGS

In order to understand the disclosure and to see how it may be carried out in practice, embodiments will now be described, by way of non-limiting examples only, with reference to the accompanying drawings, in which:

FIGS. 1A-1C are a series of visual stimuli for an eye examination in accordance with an example of the presently disclosed subject matter;

FIGS. 2A-2C are a series of visual stimuli for an eye examination in accordance with another example of the presently disclosed subject matter;

FIG. 3 is a front view of a visual stimulus for an eye examination in accordance with yet another example of the presently disclosed subject matter

FIG. 4 is a flow chart diagram illustrating a method for calculating a range of optical powers;

FIG. 5A is a flow chart diagram illustrating a method for conducting an eye examination in accordance with another example of the presently disclosed subject matter;

FIG. 5B is a flow chart diagram illustrating a method for conducting a binocular test in accordance with an example of the presently disclosed subject matter;

FIG. 6 is graph representations of eye examination results carried out in accordance with the method of FIG. 5A;

FIG. 7A is graph representations of results extracted from the graph of FIG. 6; and

FIG. 7B is graph representations of a section of the graph of FIG. 7A.

### DETAILED DESCRIPTION OF EMBODIMENTS

It is well known that the human eye can distinguish between colors by resolving a multi-color pattern as it reaches the retina. The retina includes photoreceptor cells, which are interconnected such as to form overlapping receptive fields across the retina. The photoreceptive cells can be functionally classified into two distinct categories: The ‘rods’, which are sensitive to light in the visible spectrum irrespective of its wavelength (color), and the ‘cones’, which can be further sub-classified into those sensitive to red, blue, and green light. The receptive fields on the retina consist of clusters of each type of photoreceptive cell. Patterns, such as images, are resolved when the light at differential luminance (energy) levels, with the appropriate wavelengths, arrives at a cluster of adjacent receptive fields of the same type. Importantly for the current discussion is the phenomenon whereby a cluster of overlapping receptive fields of cone receptors, sensitive to the three different wavelengths, is illuminated at roughly equal luminance levels, the perceived light lacks color and appears as a shade of gray (black at lowest luminance and white at highest luminance), whereas differential illumination of the same cluster at different wavelengths will result in a perception of color. In practice, the absence or presence of color in a visual display, or part thereof, is easily distinguishable and can be responded to with relative ease.

Thus, the ability to detect color in a light pattern consisting of multiple elements with different wavelengths depends, in part, on the following factors: the quality (sharpness) of the image generated by the pattern on the retina (i.e., the degree of overlap whether between the receptive fields on the retina which receive light from each of the elements in the pattern), and the spatial frequency of the pattern's image (i.e., the size of the image generated by each element in the pattern on the retina). The spatial frequency is determined by the physical size of the original pattern, and its apparent proximity to the eye. The sharpness of the image is affected by the accurate focusing of light on the retina by the eye's lens in conjunction with any additional corrective (or other) lenses placed in the optical path between the light source and the eye.

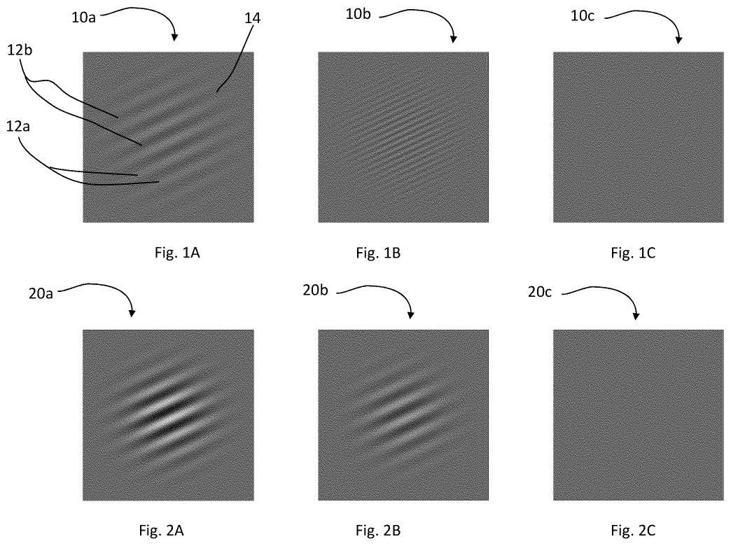

The presently disclosed subject matter is directed to a method for carrying out an eye examination on an examinee. The method includes providing a display configured for displaying a visual stimulus and forming a visual path between the display and at least one eye of the examinee. The visual stimulus which is displayed to the examinee includes a first visual element having a first color, a second visual element having a second color, and a background having a third color. For example, as shown in FIG. 1A, the visual stimulus 10 includes a first element 12a, which is a plurality of first lines in a first color, and a second element 12b, which is a plurality of second lines in a second color. The first and second lines are parallel to one another and are disposed such that the lines are alternating between the first lines 12a and the second lines 12b. The alternating first and second lines 12a and 12b are displayed over a background 14 of a third color.

The first, second and third colors are selected such that contrast between a component of the first color and the third color is the same as a contrast between a component of the second color and the third color. According to an example, the first lines 12a are orange lines, the second lines 12b are blue lines, and the background is gray. The orange color of the first element 12a, and the gray color of the background 14, are selected such that certain component of the orange color, such as a certain wavelength, has a contrast of a certain level with respect to the gray color. The desired level of contrast can be achieved for example, by providing the component of the orange color with a first intensity, and the gray color with a second intensity. The difference between the first and second intensities is the contrast level between the component of the orange and the gray color.

When using an electronic display for displaying the visual stimulus, the composition of the first, second, and third colors can be determined by using RGB parameters. In RGB terms, the example of orange, blue, and gray colors can be determined with the values (255, 165, 75) for the orange color, values (75, 165, 255) for the blue color, and values (165, 165, 165) for the gray color. As a result, the R component of the orange color and the R component of the gray color has an intensity difference of 90. Similarly, the R component of the blue color and the R component of the gray color has an intensity difference of −90. Thus, the contrast between a component of the orange and the gray is the same as the contrast between a component of the blue and the gray. In other words, the RGB components of the background have values that are determined as an average between values of corresponding RGB components of the orange and the blue.

Another example of a color composition of the first, second, and third colors is using purple, green, and gray colors, which are determined with the values (130, 28, 232) for the purple color, values (130, 232, 28) for the green color, and values (130, 130, 130) for the gray color. As a result, the G component of the purple color and the G component of the gray color has an intensity difference of 102. Similarly, the G component of the green color and the G component of the gray color has an intensity difference of −102. Thus, the contrast between a component of purple and gray is the same as the contrast between a component of green and gray.

The aim of using colored stimuli in vision tests according to the present invention is to allow for the detection of color on a uniform background by the examinee. To achieve this effect, light a pattern needs to be constructed such that two of the colors alternate in it for the purpose of detection. The uniform background must correspond to the average value of the two alternating colors. However, it was discovered that the desired effect is greatest when the uniform background is in grayscale, which requires the examinee to respond to the appearance of any color on an otherwise colorless background, rather than distinguishing between colors.

It is noted that in the above examples, the RGB components of each of the first and second colors include symmetrical values. I.e., the value of the R and B components of the orange and blue colors are the average of the G component.

Another way to express the composition of the first and second colors is by the luminance level. I.e., the average luminance level of two components of the first color is the luminance level of the third component of the first color. For example, when using a sinusoidal grating that alternates between red component and green component (i.e., sinus value of 1 corresponds to maximal luminance of red and minimal luminance of green, whereas the sinus value of −1 corresponds to opposite luminance of the two colors), the average luminance across the pattern would correspond to “0” (or half the maximal possible luminance. In this case, the luminance of the blue component should thus be kept at 0.5 of maximum luminance, with no alteration across the area of the pattern.

It is noted, however, that the RGB components of each of the first and second colors do not have such internal symmetricity, as described above. Rather, the first and second colors are selected such that the contrast value of one of the RGB components and the gray background is the same as the first color and the second color.

As explained above, the ability to detect colors in the visual stimulus depends on the properties of the visual stimulus, which affect the contrast level between the first and second elements of the visual stimulus and the background. The contrast level can depend for example by the sharpness, spatial frequency of elements in the visual stimulus and other properties.

Accordingly, the visual stimulus displayed can include a series of visual stimuli presented to the examinee in a certain order. Each of the visual stimuli in the series has a contrast of a predetermined value such that visual acuity can be assessed in accordance with the contrast level of the visual stimulus at which at least one of the first and second colors is detected.

For example, as shown in FIGS. 1A-1C, the series of visual stimuli include visual stimulus of alternating-colored lines with varying spatial frequencies. Although in the enclosed drawings the visual stimuli appear in gray scale, the various elements in the visual stimuli are presented in colors as explained hereinbelow. The visual stimulus 10b shown in FIG. 1B, has a higher spatial frequency and thus would be perceived as uniform gray (as visual stimulus 10c shown in FIG. 1C) by persons with uncorrected vision, under conditions in which a persons with corrected vision would perceive colored lines, similar to visual stimulus 10a of FIG. 1A.

According to another example, as shown in FIGS. 2A-2C, the series of visual stimuli include visual stimulus of alternating-colored lines, with varying relative luminance. The visual stimulus 20b shown in FIG. 2B, has a lower relative luminance and thus will be perceived as uniform gray (as visual stimulus 20c shown in FIG. 2C) by persons with uncorrected vision, under conditions in which persons with corrected vision will perceive colored lines, similar to visual stimulus 20a of FIG. 2A.

Thus, presenting the above series of visual stimuli facilitate assessing visual acuity. This is since when the display is out of focus due to the spherical lens' value being far from the actually required correction, the colors in the two overlapping lines will blend to form a color identical to that of the background. Thus, the examinee will not be able to detect any color. As the value of the lens approaches the required correction value, the examinee will be able to resolve and detect the colors.

It would be appreciated by those skilled in the art that the ability to detect colors would also depend on astigmatism. For that, the visual stimulus can include an orientation having an axis, and the visual stimulus is displayed at various orientations. For example, when the first and second visual elements in the visual stimulus include lines, the visual stimulus is displayed with the lines at predetermined angles with respect to the background.

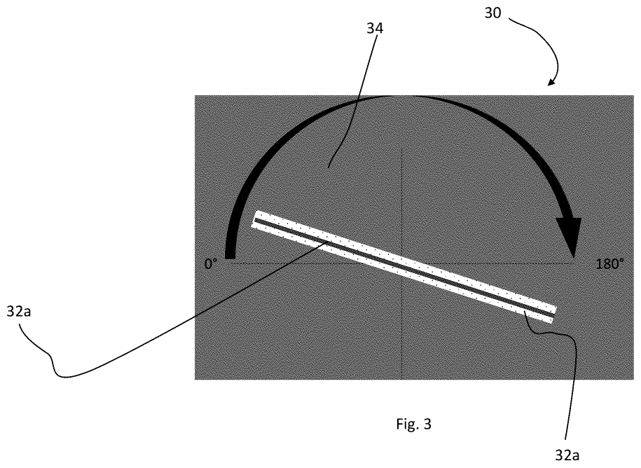

According to a further example, as shown in FIG. 3, the first element of the visual stimulus 30 includes a first line 32a of a first color (shown as dotted background) having a first width and the second element includes a second line 32b of a second color (shown as solid background) having a second width. The second line 32b is disposed on top of the first line 32a and the visual stimulus 30 includes background 34 of a third color. During the test the visual stimulus 30 is rotated with respect to the background 34, such that the first and second lines 32a and 32b are displayed at different angles.

Thus, an examinee without astigmatism will be able to detect the color of the second lines 32a throughout its apparent 180° rotation. As explained hereinbelow the required spherical correction would be detected by modifying the optical power of the lens between a maximum optical power at which the examinee detects colors and a minimum optical power at which the examinee detects colors. The midpoint of the range at which color was detected would correspond to the required spherical correction for that examinee.

For an examinee with astigmatism, as the spherical lens' value is reduced, the first or second colors will be detected along the angle perpendicular to the cylinder's axis. This is since the negative value of cylindrical aberration on the axis reduces the positive addition of the spherical lens. This process is explained herein below in detail. The use of visual stimulus 30 allows continuous rotation of the visual stimulus and receiving an indication from the examinee at which optical power colors were detected and at which angles.

According to an aspect of the present invention, the visual stimulus 30 shown in FIG. 3, can be used without colors, rather with varying contrast. I.e., the visual stimulus 30 can be configured such that the contrast between the first visual element 32a and the background 34 is the same as a contrast between the background 34 and the second visual element 32b. Although the use of colors assists in detecting vision acuity, the use of varying contrasts of the same color, such as grayscale, can also be used for detecting vision acuity. The rotation of the visual stimulus 30 with respect to the background facilitates receiving an indication from the examinee regarding detection of at least one of the first and second elements at various angles. The contrast between the elements 32a and 32b of the visual stimulus and the background 34 can be modified for detecting the threshold at which the examinee detects the elements. Furthermore, the varying axes of the elements 32a and 32b allow detecting astigmatism.

The advantage of using such visual stimulus is the ability to conduct the eye examination as a continuous process during which the visual stimulus is rotated and the optical power (or equivalence of the optical power) is modified. The examinee is only required to indicate, for example, by pressing a button when elements 32a and 32b are detected. This is contrary to known eye examinations in which the examinee is displayed discrete visual elements and is required to compare the vision of the visual elements with certain optical power and vision of the visual elements with another optical power.

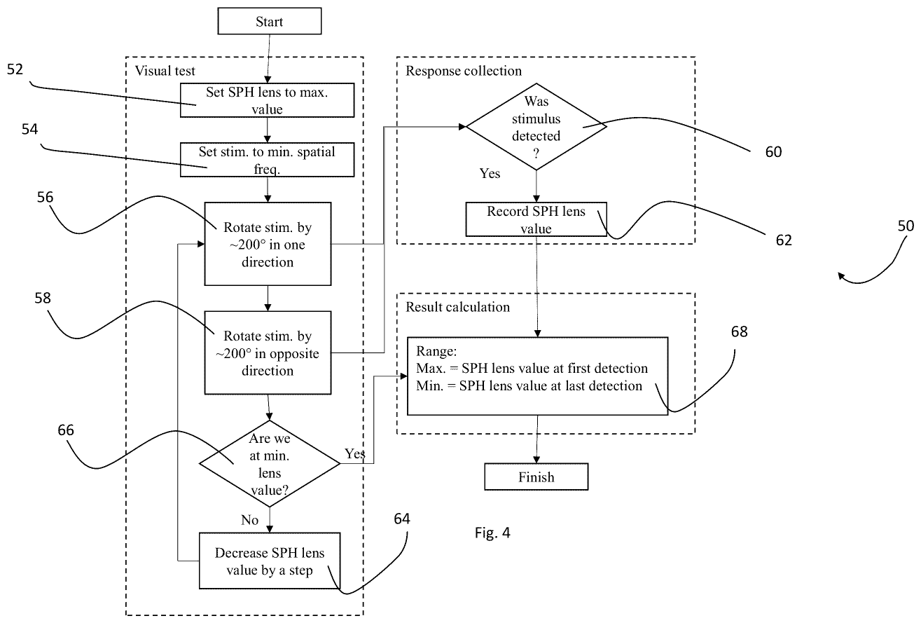

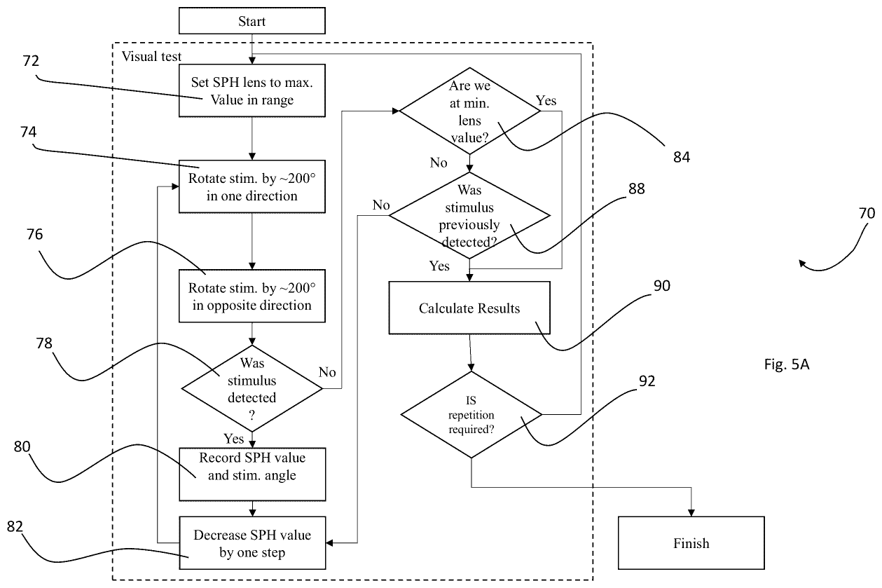

As explained hereinabove, detecting the required spherical correction can be detected by modifying the optical power of the lens between a maximum optical power at which the examinee detects colors (or the visual elements in case grayscale is used) and a minimum optical power at which the examinee detects colors. The midpoint of the range at which color was detected would correspond to the required spherical correction for that examinee. Reference is now made to FIG. 4, showing a flow chart of a method 50 for detecting the range of spherical optical power at which colors are detected. According to an example, the range detection method 50 include setting the value of the spherical power of the lens to maximum (block 52), and setting the contrast property of the visual stimulus to a minimum. As indicated above, the contrast property of the visual stimulus to a minimum can be the spatial frequency (block 54), or other elements that would affect the ability to detect elements of the visual stimulus. According to the example of FIG. 3, the ratio between the width of the first and second lines can be modified to control the contrast level between the lines and the background.

Further, the visual stimulus is rotated by approximately 200° in one direction (block 56), and then rotated by approximately 200° in the opposite direction (block 58). During the rotation of the visual stimulus, responses from the examinee are collected, and when the examinee indicates that the visual elements of the visual stimulus are detected (block 60), the value of the spherical lens used is recorded (block 62).

Following the rotation of the visual stimulus, the value of the spherical power of the lens is slightly decreased (block 64), and the rotation of the visual stimulus is repeated. Once again, if the examinee indicates that the visual elements of the visual stimulus are detected (block 60), the value of the spherical lens used is recorded (block 62).

This process is repeated until the spherical power of the lens is reduced to the minimum (block 66), at which point the data collected is analyzed. According to an example, the range is defined as a maximum value of the spherical power at the first detection a minimum value of the spherical power at the last detection (block 68). It would be appreciated that starting the detection process from the maximum spherical power facilitates eliminating errors caused by eye accommodation.

As discussed above, the required optical power can be determined as a midpoint between the calculated minimum and maximum. However, according to other examples, other parameters can be taken into consideration, such as properties of the visual stimulus, age of examinee, light conditions, etc.

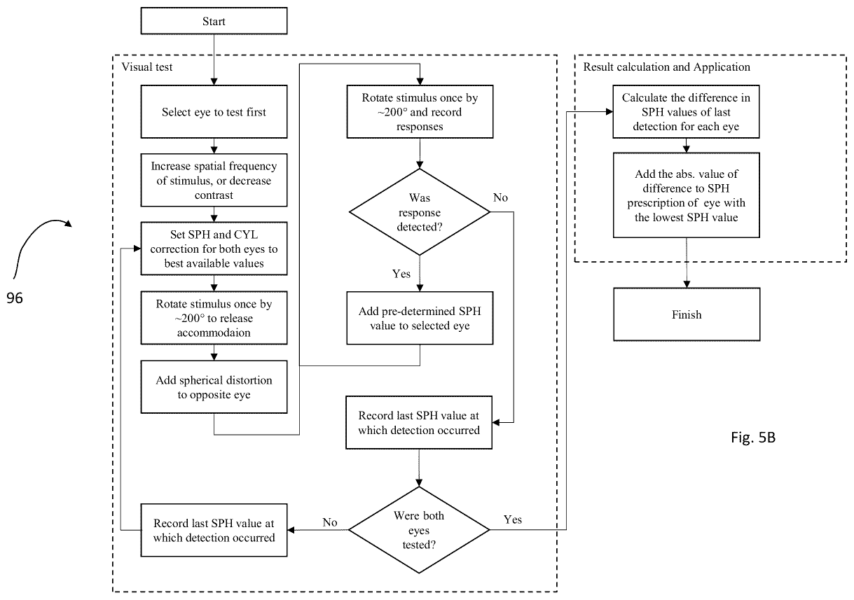

With reference to FIG. 5A, the eye examination can further include collecting data related to cylindrical correction. The method for collecting the cylindrical data 70, includes setting the optical power of the lens to the maximum power previously detected (block 72). Further, the visual stimulus is rotated by approximately 200° in one direction (block 74), and then rotated by approximately 200° in the opposite direction (block 76). During the rotation of the visual stimulus, responses from the examinee are collected, and if the examinee indicates that the visual elements of the visual stimulus are detected (block 78), the value of the spherical lens used is recorded together with the angle at which the detection occurred (block 80). It would be appreciated that detection of the angle can include detection of a range of angles. This is since the visual stimulus is continuously rotated, and the examinee indicates continuous detection along with the range of angles, for example, by continuously pressing a button so long as elements of the visual stimulus are detected.

It is noted that according to the present example, the visual stimulus is rotated in two opposite directions so as to eliminate various factors, such as response time, eye accommodation, etc.

Once the rotation in the two opposite directions is completed, the value of the optical power of the lens is decreased by one step, such as one or half of the diopter (block 82), and the rotation of the visual stimulus in two opposite directions is repeated (blocks 76 and 78), and the value of the spherical lens used is recorded together with the angle at which the detection occurred (block 80). In case during the rotation, no detection was indicated (block 84), and the optical power of the lens is not at the minimum value (block 86), and no stimulus was yet detected (block 88), the value of the optical power of the lens is decreased by one step (block 82). Otherwise, if either the lens it already at the minimum value (block 84) or the stimulus was already detected (block 88), the test is terminated.

Finally, data of all the various detection is collected and analyzed (block 90), this data set includes the angles and optical power at which the visual stimulus as detected. In other words, the data set includes rows of data wherein each row represents an instance of the test, which have a value of the spherical power, the angle at the instance, and an indication of whether or not detection of the visual stimulus was indicated. In addition, each row can further include the direction of motion of the visual stimulus.

Upon calculation of the data as described hereinbelow, it might be revealed that additional repetitions are required in order to obtain conclusive results (block 92). In such a case, the test can be repeated with the same or other visual stimulus and optical power range.

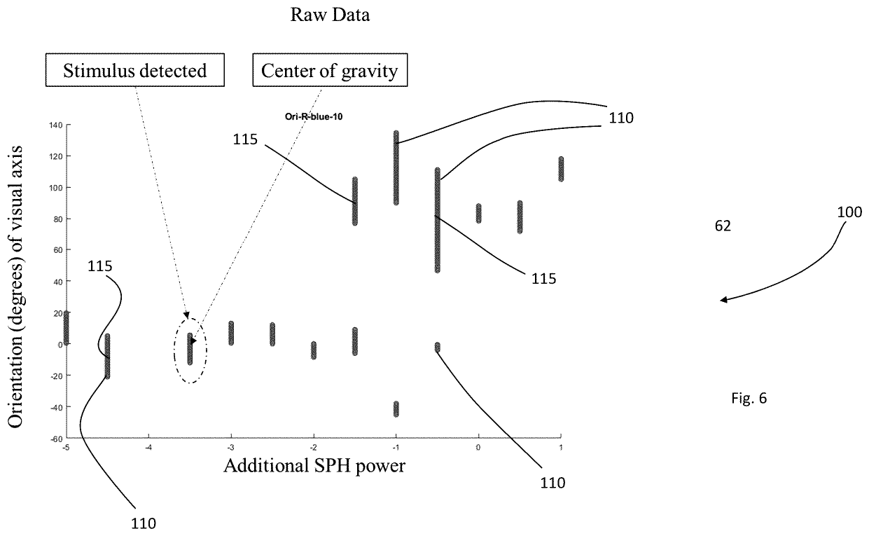

As shown in FIG. 6, the collected data can be presented in graph 100, showing subsets 110 of the collected data divided by each discrete value of the spherical power and presented as a function of the visual axis of the visual stimulus at which detection has occurred.

As shown in the graph, subsets 110 show the range of angles at which the visual stimulus was detected for each spherical power in the tested range. As shown in graph 100, for a certain spherical power, more than one subset can be detected, such as shown at −0.5 diopters. In such a case, the longest continuous sequence of detection can be selected, and the shorter one is removed.

Further, the mid-point 115 of each subset is detected, which can be calculated as the median angle in the range of angles at which the visual stimulus was detected for each spherical power. Alternatively, the mid-point can be the middle between the maximum and minimum angel, or as a center of gravity, in case the range angles is tested more than one time.

Next, if the mid-points 115 of all subsets 110 are aligned on the same visual axis, it can be deduced that no astigmatism was detected. In this case, the required spherical correction is calculated as a weighted median spherical power value of all-spherical power values at which detections occurred. It is noted that the weighting reflects accommodative responses of the eye at a lower value of the spherical power of the range.

As shown in the graph, for an examinee with a astigmatism, the mid-points 115 would be aligned along two visual axes. In case the two alignments have a difference of roughly 90° angle, one at the higher spherical power values and the other at the lower spherical power values, then astigmatism is detected.

According to an example, artifact correction can be calculated. This can be carried out if each subset, as described above, contains values from only one rotation direction. The midpoint of the subset can be skewed in the direction of the motion due to the delays related to response times to the apparent appearance/disappearance of the stimulus. In this case, the artifact can be approximately corrected by averaging with neighboring samples.

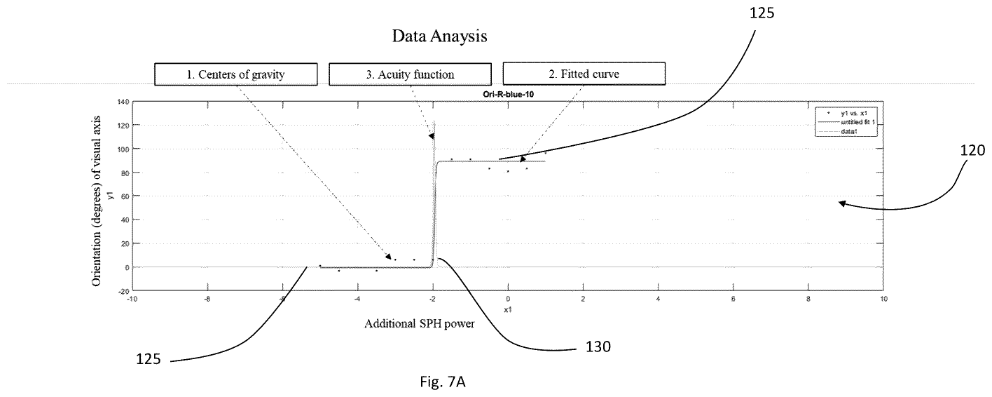

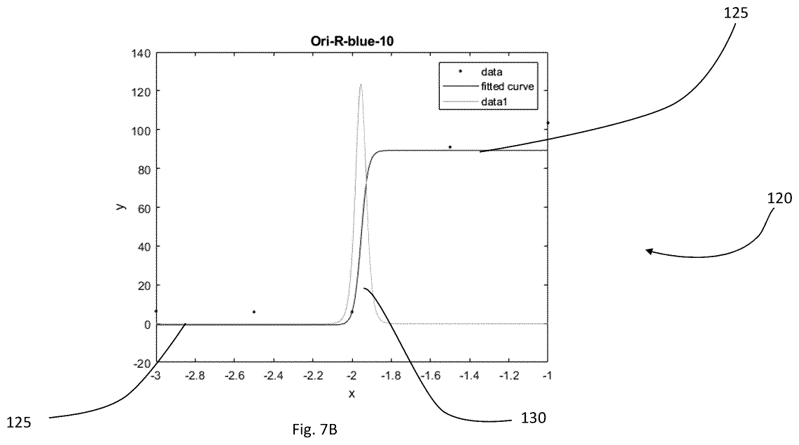

Referring now to FIGS. 7A and 7B, the data is fitted to a sigmoid formula and be presented as a curve 120 with an amplitude of 90, wherein the angle value of the mid-points is a function of the spherical power value at which the midpoint was measured. Such fitting allows extracting a parameter of “Goodness of fit” (R²) of the fitting. The goodness of fit value can be compared to a pre-set value or pre-set threshold, such as 0.4 or 0.6, which indicates a certainly required accuracy of the results. If this parameter is not above the threshold, the tests can be repeated in order to collect more data. Otherwise, failure to meet the required Goodness of fit value may indicate that the examined eye does not have astigmatism. In such case the test may be repeated to obtain more data and the calculation can be carried out again. If the Goodness of fit value is still below the required threshold, the data may indicate that the examinee has other vision impairments.

It is noted however that the data collected during the eye examination can be used for detecting higher order of aberrations, such as gradient etc. In such case instead of using a sigmoid formula, other formulas can be used for detecting the specific aberration.

Further, the point on the curve 120 with the steepest gradient can be detected. This point represents the value of the required spherical correction. In addition, the visual angles at each asymptote 125 of the curve are detected. The values of these two visual angles are 90° to each other, and these values represent the cylinder axis. I.e., the cylinder axis is the angle at which detection occurred at the lower spherical power values, and the counter-axis is at 90° to this cylinder axis.

Next, the cylinder optical value is determined by calculating the derivative function of curve 120. The derivative function provides a Gaussian-like curve 130, as best shown in FIG. 7B. In case the Gaussian-like curve 130 is inverse (i.e., there is a trough instead of a peak) the function is multiplied by a negative. Finally, the distribution of the Gaussian-like curve 130 is calculated to provide a variance value. This variance value represents the cylinder optical value, i.e., the difference in the added power between the cylinder axis and the counter-axis.

Finally, after determining the required optical correction for each eye, a binocular test can be conducted to consider the vision in both eyes simultaneously. As shown in FIG. 5B, the binocular test 95 can be carried out by implementing the rotation of the visual stimulus, as carried out during the eye examination of each eye. This example provides detailed steps to be taken as described in the flow chart. It would be appreciated that other methods for a binocular test could be implemented as well.

Although the above method makes reference to setting optical power of the lens at various values so as to detect the required spheric correction of the examinee, according to an example, the optical power can be simulated by imaging means. For example, the method can include displaying the visual stimulus on a display in various manners which simulating a range of distances. Each distance can be configured to correspond to vision through a lens having a certain optical power. Such display can be incorporated, for example, in a virtual reality apparatus, or other display devices such as a smartphone etc. in addition, the display and the above method can be implemented in a standard phoropter device and can be carried out in conjunction with other eye vision tests.

The visual stimulus can have other characteristics corresponding to vision through a lens having a certain spherical power. For example, each visual stimulus can have a certain blurring level corresponding to vision via a predetermined spherical power. This way, instead of using a lens and physically modifying the optical power, the visual stimulus can be modified to include a blurring level corresponding to the desired optical power. Similarly, the characteristics of the visual stimulus can include resolution level, which corresponds to vision via a predetermined spherical power.

Furthermore, according to the present invention, the data collected in vision tests can be recorded in a database and can be used for improving the analysis of future tests. For example, the manner in which the optical range is detected or the manner in which color composition is determined can be optimized by the collected data. Furthermore, the data can include the age of the examinee and other information which can be used for improving the analysis of future tests, for example, by factoring response time at each age group or sensitivity to specific color composition. Such data can be integrated with machine learning capabilities and can facilitate the optimization of the examination method. This way, the method can be implemented by a computerized system having an automatic process dictated by the indications provided by the examinee and by data collected in previous eye examinations.

Those skilled in the art to which the presently disclosed subject matter pertains will readily appreciate that numerous changes, variations, and modifications can be made without departing from the scope of the invention, mutatis mutandis.

1. A method for carrying out an eye examination on an examinee, the method comprising:
providing a display configured for displaying a visual stimulus;
forming a visual path between said display and at least one eye of the examinee;
displaying on said display at least one visual stimulus including a first visual element having a first color, a second visual element having a second color, and background having a third color, wherein said first, second and third colors are selected such that contrast between a component of said first color and said third color is the same as a contrast between a component of said second color and said third color;
receiving an indication from the examinee regarding detection of at least one of said first and second colors;
assessing vision of said at least one eye in accordance with said indication and said contrast.

2. The method according to claim 1 wherein said step of displaying at least one visual stimulus includes displaying a series of visual stimuli wherein said contrast has a predetermined value for each one of the visual stimulus, and wherein said step of assessing vision includes assessing the contrast of the visual stimulus at which at least one of said first and second colors is detected.

3. The method according to claim 2 wherein each one of said visual stimulus includes an orientation having an axis, and wherein said step of displaying visual stimulus includes displaying said visual stimulus at various orientations and said step of assessing vision further includes assessing the axis of said orientation of a visual stimulus of which at least one of said first and second colors are detected.

4. The method according to claim 3 wherein said first and second visual elements includes lines, wherein for each one of said visual stimulus said orientation includes disposing said lines at a predetermined angle with respect to said background.

5. The method according to claim 4 wherein said series of visual stimuli includes a plurality of visual stimulus such that said lines are disposed at a plurality of angles between 0°-180°.

6. The method according to claim 1 wherein said first, second and third colors are selected such that difference between intensity of said component of said first color and intensity of said third color is the same as difference between intensity of said component of said second color and intensity of said third color.

7. The method according to claim 1 wherein said first, second and third colors are determined with RGB parameters, and are selected such that for said first color a first parameter of the RGB parameters includes a first value, and second parameter of the RGB parameters includes a second value, and for said second color a first parameter of the RGB parameters includes the second value and second parameter of the RGB parameters includes the first value, and wherein for the third color a first and second parameter of the RGB parameters includes a third value which is an average between said first and second values.

8. The method according to claim 1 wherein said first and second elements are parallel lines disposed such that said lines alternating between said first color and said second color.

9. The method according to claim 1 wherein said first element includes a first line in said first color having a first width and said second element includes a second line in said second color having a second width, wherein said second line is disposed on top of said first line and wherein said second width is smaller than said first width.

10. The method according to claim 1, wherein said visual path includes a lens having a spherical power, and wherein said step of assessing vision is in accordance with said spherical power.

11. The method according to claim 11, further comprises modifying said spherical power in accordance with said indication and wherein said step of displaying said visual stimulus includes displaying a plurality of visual stimulus for one or more spherical powers.

12. The method according to claim 1, wherein said visual stimulus includes displaying a plurality of visual stimulus each of which having characteristics corresponding to vision through a lens having a certain spherical power.

13. The method according to claim 12 wherein said characteristics include a blurring level corresponding to vision via a predetermined spherical power.

14. The method according to claim 12 wherein said characteristics include optical resolution level corresponding to vision via a predetermined spherical power.

15. The method according to claim 3 further includes collecting dataset including axis of each visual stimulus, value of a spherical power, and said indication, said dataset is manipulated by a selected mathematical formula to obtain a required optical correction.

16. A method for carrying out an eye examination on an examinee, the method comprising:
providing a display configured for displaying a visual stimulus;
forming a visual path between said display and at least one eye of the examinee;
displaying on said display at least one visual stimulus including a first visual element, a second visual element, and background, wherein contrast between said first visual element and said background is the same as contrast between said background and second visual element;
rotating said first and second elements with respect to said background modifying thereby orientation of said first and second visual elements with respect to the background;
receiving an indication from the examinee regarding detection of at least one of said first and second elements;
assessing vision of said at least one eye in accordance with said indication and said contrast.

17. The method of claim 16 wherein said orientation has an axis, and wherein said step of displaying visual stimulus includes displaying said first and second visual elements at various orientations and said step of assessing vision further includes assessing the axis of said orientation when said first or second visual elements are detected.

18. The method of claim 16 further comprises modifying level of said contrast and wherein said step of assessing vision further includes assessing level of said contrast when said first or second visual elements are detected.
